(19) United States
(12) Patent Application Publication
(10) Pub. No.: US 2009/0326513 A1
(43) Pub. Date: Dec. 31, 2009

(54) DEVICE FOR PLACING AN OCCLUSION REMOVING STRUCTURE IN AN OPEN-ENDED TUBULAR STRUCTURE

(76) Inventors: Harvey L. Deutsch, Los Angeles, CA (US); To V. Pham, Trabuco Canyon, CA (US)

Correspondence Address: SHELDON MAK ROSE & ANDERSON PC, 100 Corson Street, Third Floor, PASADENA, CA, 91103-3842, US

(21) Appl. No.: 11/573,262

(22) PCT Filed: Jan. 26, 2007
(86) PCT No.: PCT/US07/61132

§ 371 Date: Feb. 5, 2007

Related U.S. Application Data
(60) Provisional application No. 60/764,232, filed on Jan. 31, 2006.

(51) Int. Cl. A61M 25/00

(52) U.S. Cl. 604/540

(57) ABSTRACT

A device and method for placing an occlusion removing structure into a tubular structure with an open distal end, where placing results in the distal end of the occlusion removing structure being positioned at or near the distal end of the tubular structure as disclosed in this disclosure.

### CROSS-REFERENCE TO RELATED APPLICATION

The present Application claims the benefit of U.S. Provisional Patent Application 60/764,232 titled “Device for Placing an Occlusion Removing Structure in an Open-ended Tubular structure,” filed Jan. 31, 2006, the contents of which are incorporated in this disclosure by reference in their entirety.

### BACKGROUND

There are a large variety of tubular structures comprising a central axial lumen and an open distal end used in medical applications, as well as in non-medical commercial and industrial applications. Examples of such tubular structures used in medical applications include catheters, tubular structures and stents. In many such applications, the tubular structure is prone to collecting occlusive debris within the central lumen of the tubular structure that occludes the tubular structure, causing a decrease or cessation of function of the tubular structure, and necessitating that the occlusion be removed or that the tubular structure be replaced.

In medical applications in particular, replacing a tubular structure can be very costly, and can put a patient at increased risk of infection. Therefore, it is often preferable to remove the occlusion from within the central lumen of the tubular structure while maintaining the tubular structure in position. In some applications, the occlusion can be removed by flushing the central lumen of the tubular structure with a liquid, thereby moving the occlusion distally and out of the tubular structure. In many applications, however, flushing the occlusion distally is not appropriate, such as, for example, when using a tubular structure to tubular structure a cavity or space within the human body. Therefore, in some applications, an occlusion removing structure is placed within the tubular structure and, when the tubular structure becomes occluded, the occlusion removing structure is withdrawn from the tubular structure proximally, thereby removing the occlusion proximally rather than distally and also maintaining the tubular structure in position. Placing an occlusion removing structure in the tubular structure can be performed before the tubular structure becomes occluded, or can be performed after an occlusion has been removed from the tubular structure, such as for example to replace an occlusion removing structure that has been removed so that another occlusion can be removed from the tubular structure if the tubular structure becomes occluded again. It is difficult, however, to place an occlusion removing structure into a tubular structure with an open distal end in a proper position, particularly where the exact length of the tubular structure is unknown, because the occlusion removing structure can be advanced too far distally, where the distal end of the occlusion removing structure extends through the open distal end of the tubular structure and into the cavity or space being drained, or the occlusion removing structure can be advanced insufficiently distally, where the distal end of the occlusion removing structure is significantly proximal to the distal end of the tubular structure, thereby allowing an occlusion to accumulate between the distal end of the occlusion removing structure and the distal end of the tubular structure.

Therefore, there is a need for a device for placing an occlusion removing structure into a tubular structure with an open distal end, particularly where the exact length of the tubular structure is unknown, where placing results in the distal end of the occlusion removing structure being positioned at or near the distal end of the tubular structure. Further, there is a need for a method for placing an occlusion removing structure into a tubular structure with an open distal end, particularly where the exact length of the tubular structure is unknown, where placing results in the distal end of the occlusion removing structure being positioned at or near the distal end of the tubular structure.

### SUMMARY

According to one embodiment of the present invention, there is provided a device for placing an occlusion removing structure into a tubular structure with an open distal end. The device comprises, a) a proximal end, a distal end, and an intermediate portion between the proximal end and the distal end; b) an occlusion removing structure comprising a proximal end and a distal end, where the proximal end of the occlusion removing structure comprises a first connector, and where the distal end of the occlusion removing structure comprises an elongated tubular mesh comprising a proximal end, a distal end, an axial length defined by the distance between the proximal end and the distal end, an outer surface, an inner surface, and a central lumen defined by the inner surface and further comprising a central lumen cross-sectional area perpendicular to the axial length; c) a distracting structure for distracting the occlusion removing structure from a radially contracted and axially expanded first state to a radially expanded and axially contracted second state, where the distracting structure comprises a proximal end and a distal end, where the proximal end of the distracting structure comprises a second connector configured to mate with the proximal end of the first connector, and where the distal end of the distracting structure comprises an elongated tube comprising a proximal end, a distal end, an axial length defined by the distance between the proximal end and the distal end, an outer surface defining an outer circumference, an inner surface, and a central lumen defined by the inner surface; and d) a positioning structure for positioning the distal end of the occlusion removing structure, where the positioning structure comprises a proximal end and a distal end, where the proximal end of the positioning structure comprises a third connector configured to mate with the proximal end of the second connector, where the distal end of the positioning structure comprises a catheter comprising a proximal end, a distal end, an axial length between the proximal end and the distal end, an outer surface, an inner surface, and an inflation lumen defined by the inner surface, and where the catheter further comprises an open proximal end and a cross-sectional area perpendicular to the axial length, and where the catheter further comprises an inflatable balloon at or near the distal end of the catheter, where the inflatable balloon has a maximum circumference when inflated, and where the outer surface of the catheter has a circumference that is smaller than the circumference of the inner surface of the elongated tube of the distracting structure, thereby permitting the catheter to fit within the elongated tube and to slide axially with respect to the elongated tube. The central lumen cross-sectional area in the first state is a first central lumen cross-sectional area, and the axial length in the first state is a first axial length. The central lumen cross-sectional area in the second state is a second central lumen cross-sectional area, and the axial length in the second state is a second axial length. The first central lumen cross-sectional area is less than the second central lumen cross-sectional area, and the first axial length is greater than the second axial length.

In one embodiment, the device further comprises a central stiffening structure. In another embodiment, the tubular mesh comprises between 5 and 50 strands. In another embodiment, the first central lumen cross-sectional area is between 1 mm² and 10 mm². In another embodiment, the first axial length is between 10 cm and 100 cm. In another embodiment, the second central lumen cross-sectional area is between 10 mm² and 100 mm². In another embodiment, the second axial length is between 5 cm and 50 cm. In another embodiment, the distal end of the tubular mesh comprises a cap with a central axial lumen with a cross-sectional area sufficient to permit the positioning structure to pass through the central axial lumen of the cap. In another embodiment, the proximal end of the elongated tube is integrally joined to the second connector. In another embodiment, the outer circumference of the elongated tube is less than the circumference of the inner surface of the tubular mesh when the tubular mesh is in the first state, thereby permitting the elongated tube to fit within the tubular mesh when the device is in the first state. In another embodiment, the catheter further comprises a blunt-end, flexible, atraumatic tip at the distal end of the catheter. In another embodiment, the distal end of the tubular mesh comprises a cap with a central axial lumen with a cross-sectional area sufficient to permit the positioning structure to pass through the central axial lumen of the cap, and where the axial length of the catheter is sufficient such that, when the device is assembled, the inflatable balloon extends through the central axial lumen of the cap.

In another embodiment, the device further comprises a central stiffening structure comprising a proximal end and a distal end. In another embodiment, the device comprises a central stiffening structure comprising a proximal end and a distal end, where the proximal end comprises a sealing member configured to mate with the proximal end of the third connector, thereby sealing the proximal end of the central inflation lumen. In a preferred embodiment, the distal end of the central stiffening structure comprises a rod comprising a proximal end, a distal end, an axial length between the proximal end and the distal end, and a surface comprising a circumference, where the proximal end of the rod is connected to the sealing member, and where the surface of the rod has a circumference that is smaller than the circumference of the inner surface of the catheter, thereby permitting the rod to fit within the inflation lumen of the catheter.

According to another embodiment, of the present invention, there is provided a device for placing an occlusion removing structure into a tubular structure with an open distal end. The device comprises a) means for removing an occlusion; b) means for positioning the distal end of the means for removing an occlusion; and c) means for distracting the means for removing an occlusion from a radially contracted and axially expanded first state to a radially expanded and axially contracted second state.

According to another embodiment of the present invention, there is provided a kit for placing an occlusion removing structure in an open-ended tubular structure comprising a device according to the present invention, and further comprising instructions on how to use the device.

According to another embodiment of the present invention, there is provided a method of placing an occlusion removing structure into a tubular structure with an open distal end, where placing results in the distal end of the occlusion removing structure being positioned at or near the distal end of the tubular structure. The method comprises a) selecting a tubular structure comprising a central lumen and comprising an open distal end with an internal circumference; b) providing a device for placing an occlusion removing structure in an open-ended tubular structure according to the present invention; c) inserting the distal end of the device into the proximal end of the tubular structure; d) advancing the distal end of the device through the central lumen of the tubular structure until the inflatable balloon at the distal end of the catheter has advanced completely through the open distal end of the tubular structure; e) inflating the inflatable balloon until the maximum circumference of the balloon exceeds the internal circumference of the open distal end of the tubular structure; f) retracting the proximal end of the device proximally until the inflatable balloon contacts the distal end of the tubular structure, thereby limiting any further proximal retraction of the device, and thereby positioning the distal end of the occlusion removing structure at or near the distal end of the tubular structure; g) deflating the inflatable balloon, and withdrawing the positioning structure from the tubular structure while maintaining the occlusion removing structure in position within the tubular structure; and h) withdrawing the distracting structure from the tubular structure while maintaining the occlusion removing structure in position within the tubular structure, thereby causing the occlusion removing structure to change from the radially contracted and axially expanded first state to the radially expanded and axially contracted second state.

In one embodiment, the device provided further comprises a central stiffening structure comprising a proximal end and a distal end, where the proximal end comprises a sealing member configured to mate with the proximal end of the third connector, thereby sealing the proximal end of the central inflation lumen, and where the method further comprises disconnecting the sealing member from the third connector to access the inflation lumen of the catheter to inflate the inflatable balloon. In another embodiment, the method further comprises withdrawing the central stiffening structure from the tubular structure. In another embodiment, the tubular structure is selected from the group consisting of a biliary stent, a biliary tube, a chest tube, a decompression catheter, a feeding tube, a gastrostomy tube, a jejunostomy tube, a mediastinal tube, a nasogastric tube, a nephrostomy catheter, a percutaneous tubular drainage catheter, a peritoneal dialysis catheter, a vascular catheter, and a ventriculostomy tube. In another embodiment, the method further comprises placing the tubular structure within a space or cavity. In another embodiment, the space or cavity is selected from the group consisting of the abdominal cavity, the bladder, the intestines, the intracranial cavity, the mediastinum, the nasal passages, the stomach, the renal pelvis and the ureter. In a preferred embodiment, the space or cavity is partially or totally created during a surgical procedure on the back, breast, chest, head, hip or vertebral column. In one embodiment, the proximal end of the tubular mesh extends proximally of the proximal end of the tubular structure, and where the method further comprises removing at least some of the proximal end of the tubular mesh that extends proximally of the proximal end of the tubular structure. In another embodiment, the method further comprises withdrawing the occlusion removing structure from the tubular structure after the tubular structure becomes occluded with an occluding material, thereby unoccluding the tubular structure.

### FIGURES

These and other features, aspects and advantages of the present invention will become better understood with regard to the following description, appended claims, and accompanying figures where:

FIG. 1 is a partial top perspective view of one embodiment of a device according to the present invention for placing an occlusion removing structure into a tubular structure with an open distal end;

FIG. 2 is an exploded, top perspective view of the proximal end of the device shown in FIG. 1;

FIG. 3 is a partial, top perspective view of one embodiment of an occlusion removing structure (first component) of the device shown in FIG. 1;

FIG. 4 is a partial, cutaway, top perspective view of one embodiment of a distracting structure (second component) of the device shown in FIG. 1 for distracting the occlusion removing structure shown in FIG. 3;

FIG. 5 is a partial, cutaway, top perspective view of one embodiment of a positioning structure (third component) of the device shown in FIG. 1 for positioning the distal end of the occlusion removing structure shown in FIG. 3; and

FIG. 6 is a partial, top perspective view of one embodiment of an optional central stiffening structure (fourth component) of the device shown in FIG. 1.

### DESCRIPTION

According to one embodiment of the present invention, there is provided a device for placing an occlusion removing structure into a tubular structure with an open distal end, where placing results in the distal end of the occlusion removing structure being positioned at or near the distal end of the tubular structure. According to another embodiment of the present invention, there is provided a method for placing an occlusion removing structure into a tubular structure with an open distal end, where placing results in the distal end of the occlusion removing structure being positioned at or near the distal end of the tubular structure. In one embodiment, the length of the tubular structure is unknown. In a preferred embodiment, the method comprises providing a device according to the present invention. The device and method prolong the useful life of a tubular structure, such as a medical tubular structure, and obviate the need for replacing the tubular structure when it becomes occluded, thereby decreasing the cost associated with tubular structure replacement, and the risk of patient morbidity associated with tubular structure replacement when the tubular structure is a medical tubular structure. Though the device of the present invention is presented primarily in the context of use with a medical tubular structure in this disclosure, the device can also be used for other purposes, as will be understood by those with skill in the art with reference to this disclosure. The device and method will now be presented in detail.

As used in this disclosure, the term “comprise” and variations of the term, such as “comprising” and “comprises,” are not intended to exclude other additives, components, integers or steps.

As used in this disclosure, two elements of a device are “integral” if they are joined together in a manner that does not allow separation of the two elements from one another by the user of the device without cutting through or destroying at least one of the elements.

As used in this disclosure, two elements of a device are “non-integral” if they are joined together in a manner to allow separation of the two elements from one another by the user of the device without cutting through or destroying either of the elements.

As used in this disclosure, the term “occlude” and variations of the term, such as “occluded,” “occluding,” and “occlusion” means a mass or clog of occluding material within the central lumen of a tubular structure, which either partially or completely decreases the function of a tubular structure. As will be understood by those with skill in the art with reference to this disclosure, debris being aspirated from inside a blood vessel left after removal of an embolus or plaque from the blood vessel wall by a therapy catheter does not decrease the function of an aspiration catheter aspirating the debris and, therefore, the debris within the aspiration catheter is not an occlusion within the aspiration catheter.

As used in this disclosure, the term “at or near the distal end of the tubular structure” means that the distal end of the occlusion removing structure is positioned within the distal most 10% of the axial length of the tubular structure. For example, if the axial length of the tubular structure is 10 cm, then after placing the occlusion removing structure, the distal end of the occlusion removing structure is positioned between 9 cm and 10 cm from the proximal end of the tubular structure, that is, the distal end of the occlusion removing structure is positioned within 1 cm of the distal end of the tubular structure.

All dimensions specified in this disclosure are by way of example only and are not intended to be limiting. Further, the proportions shown in these Figures are not necessarily to scale. As will be understood by those with skill in the art with reference to this disclosure, the actual dimensions of any device or part of a device disclosed in this disclosure will be determined by its intended use.

The devices of the present invention and their component parts comprise any suitable material for the intended purpose of the device, as will be understood by those with skill in the art with reference to this disclosure. For example, when used as a medical tubular structure, the device will usually comprise one or more than one biocompatible material capable of being sterilized by commonly used medical sterilization techniques.

The devices of the present invention and their component parts can be constructed according to standard techniques, as will be understood by those with skill in the art with reference to this disclosure.

As will be understood by those with skill in the art with reference to this disclosure, the device and method of the present invention can be used for a variety of both medical and non-medical uses. Examples of medical uses include using the device to place an occlusion removing structure within a tubular structure selected from the group consisting of a biliary stent, a biliary tube, a chest tube, a decompression catheter, a feeding tube, a gastrostomy tube, a jejunostomy tube, a mediastinal tube, a nasogastric tube, a nephrostomy catheter, a percutaneous tubular drainage catheter, a peritoneal dialysis catheter, a vascular catheter, and a ventriculostomy tube.

According to one embodiment of the present invention, there is provided a device for placing an occlusion removing structure into a tubular structure with an open distal end, where placing results in the distal end of the occlusion removing structure being positioned at or near the distal end of the tubular structure. The device comprises means for removing an occlusion, means for positioning the distal end of the means for removing an occlusion, and means for distracting the means for removing an occlusion from a radially contracted and axially expanded first state to a radially expanded and axially contracted second state. The structures disclosed in this disclosure that correspond to the means for removing an occlusion, the means for positioning the distal end of the means for removing an occlusion, and the means for distracting the means for removing an occlusion will be understood by those with skill in the art with reference to this disclosure. In general, as disclosed in this disclosure, the means for removing an occlusion corresponds to the occlusion removing structure (first component) of the device, the means for distracting the means for removing an occlusion corresponds to the distracting structure (second component) of the device, and the means for positioning the distal end of the means for removing an occlusion corresponds to the positioning structure (third component) of the device.

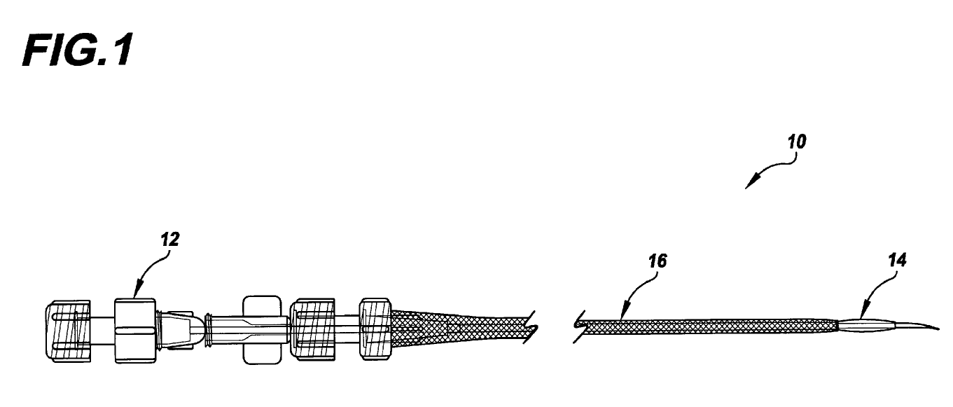

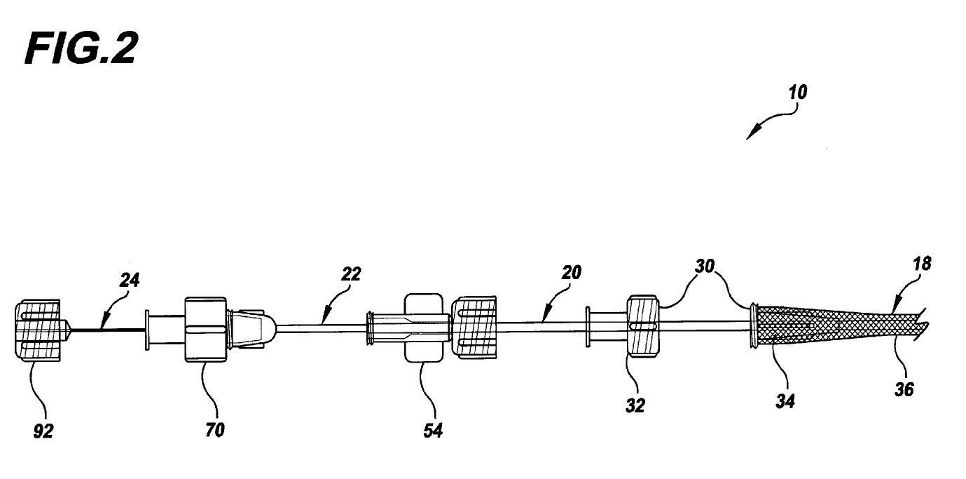

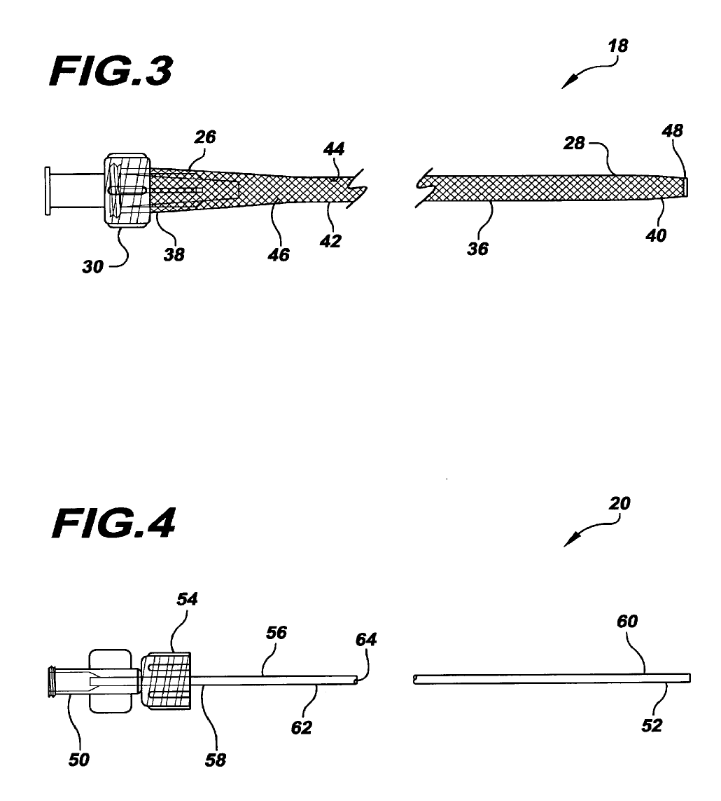

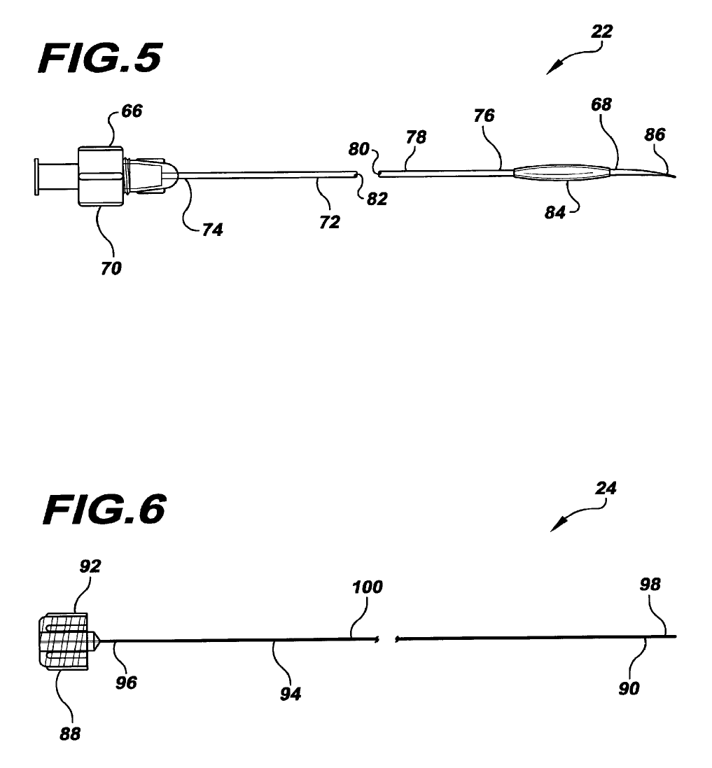

Referring now to FIG. 1, FIG. 2, FIG. 3, FIG. 4, FIG. 5 and FIG. 6, there are shown, respectively, a partial top perspective view of one embodiment of a device according to the present invention for placing an occlusion removing structure into a tubular structure with an open distal end, where placing results in the distal end of the occlusion removing structure being positioned at or near the distal end of the tubular structure (FIG. 1); an exploded, top perspective view of the proximal end of the device shown in FIG. 1 (FIG. 2); a partial, top perspective view of one embodiment of an occlusion removing structure (first component) of the device shown in FIG. 1 (FIG. 3); a partial, cutaway, top perspective view of one embodiment of a distracting structure (second component) of the device shown in FIG. 1 for distracting the occlusion removing structure shown in FIG. 3 (FIG. 4); a partial, cutaway, top perspective view of one embodiment of a positioning structure (third component) of the device shown in FIG. 1 for positioning the distal end of the occlusion removing structure shown in FIG. 3 (FIG. 5); and a partial, top perspective view of one embodiment of an optional central stiffening structure (fourth component) of the device shown in FIG. 1 (FIG. 6). As can be seen, in one embodiment of the present invention, the device 10 comprises a proximal end 12, a distal end 14, and an intermediate portion 16 between the proximal end 12 and the distal end 14. The device 10 further comprises three components, an occlusion removing structure 18 (first component), a distracting structure 20 (second component) for distracting the occlusion removing structure 18 from a radially contracted and axially expanded first state to a radially expanded and axially contracted second state, and a positioning structure 22 (third component) for positioning the distal end of the occlusion removing structure. Optionally, as will be disclosed further below, the device 10 further comprises a central stiffening structure 24 (fourth component). The occlusion removing structure 18, distracting structure 20, positioning structure 22, and when present, central stiffening structure 24 are all non-integral with respect to each other.

Referring now to FIG. 1, FIG. 2 and FIG. 3, the occlusion removing structure 18 comprises a proximal end 26 and a distal end 28. The proximal end 26 of the occlusion removing structure 18 comprises a first connector 30. In one embodiment, as shown, the first connector 30 comprises a proximal section 32 and a distal section 34, such as a separable, non-integral, two-piece, luer lock-type connector as shown, where the proximal section 32 comprises a proximal “male” extension with a distal “female” fitting comprising a threaded flange, and the distal section 34 comprises a distally tapered, “male” fitting with a threaded collar on the proximal end of the male fitting, and where the threaded collar of the male fitting is configured to mate with the threaded flange of the female fitting. The first connector 30 can, however, be any suitable connector, as will be understood by those with skill in the art with reference to this disclosure.

The distal end 28 of the occlusion removing structure 18 comprises an elongated tubular mesh 36 comprising a proximal end 38, a distal end 40, an axial length defined by the distance between the proximal end 38 and the distal end 40, an outer surface 42, an inner surface 44, and a central lumen defined by the inner surface 44 and comprising a central lumen cross-sectional area perpendicular to the axial length.

In a preferred embodiment, the tubular mesh 36 comprises a plurality of strands 46 forming a mesh, where each of the plurality strands 46 crisscross a plurality of other strands 46, rather than comprising a plurality of strands that are merely spaced apart from one another or parallel to one another. In one embodiment, the tubular mesh 36 comprises between 5 and 50 strands 46. In another embodiment, the tubular mesh 36 comprises between 10 and 20 strands 46. In another embodiment, the tubular mesh 36 comprises between 12 and 15 strands 46.

The proximal end 38 of the tubular mesh 36 can be non-integrally joined to the first connector 30, such as for example, by being engaged between the distal end of the proximal section 32 of the first connector 30 and the proximal end of the distal section 34 of the first connector 30; or the proximal end 38 of the tubular mesh 36 can be integrally joined to the first connector 30, such as for example by either mechanical means or by chemical means, such as for example by an adhesive such as an epoxy, or both by mechanical and by chemical means, or by any other suitable means, as will be understood by those with skill in the art with reference to this disclosure.

The tubular mesh 36 exists in two states, a radially contracted and axially expanded first state where the central lumen cross-sectional area is a first central lumen cross-sectional area, and the axial length is a first axial length; and a radially expanded and axially contracted second state where the central lumen cross-sectional area is a second central lumen cross-sectional area, and the axial length is a second axial length; and where the first central lumen cross-sectional area is less than the second central lumen cross-sectional area, and the first axial length is greater than the second axial length.

In one embodiment, the first central lumen cross-sectional area is between 1 mm² and 10 mm². In another embodiment, the first central lumen cross-sectional area is between 2 mm² and 5 mm². In another embodiment, the first axial length is between 10 cm and 100 cm. In another embodiment, the first axial length is between 20 cm and 60 cm. In another embodiment, the second central lumen cross-sectional area is between 10 mm² and 100 mm². In another embodiment, the second central lumen cross-sectional area is between 15 mm² and 50 mm². In another embodiment, the second central lumen cross-sectional area is between 20 mm² and 30 mm². In another embodiment, the second axial length is between 5 cm and 50 cm. In another embodiment, the second axial length is between 10 cm and 30 cm.

In one embodiment, the distal end 40 of the tubular mesh 36 comprises a cap 48 with a central axial lumen. In a preferred embodiment, the distal end 40 of the tubular mesh 36 is integrally joined to the cap 48. The central axial lumen of the cap 48 has a configuration and cross-sectional area sufficient to permit the positioning structure 22 to pass through the central axial lumen of the cap 48. Integral joining of the distal end 40 of the tubular mesh 36 to the cap 48 can be accomplished by either mechanical means or by chemical means, such as for example, by an adhesive, such as for example an epoxy, or both by mechanical and by chemical means, or by other suitable means such as spot welding or laser welding, as will be understood by those with skill in the art with reference to this disclosure.

In another preferred embodiment, the strands 46 of the tubular mesh 36 comprise a material that can be easily cut using a pair of surgical scissors or similar instrument to a desired length as appropriate for the intended use, such as for example, a cobalt-chromium-nickel-molybdenum-iron alloy specified by ASTM F1058 and ISO 5832-7, stainless steel or a ferrous alloy containing cobalt, chromium, nickel, molybdenum, manganese, carbon, and beryllium, such as Elgiloy®. To effect this purpose, in one embodiment, the strands 46 of the tubular mesh 36 comprise a material having a tensile strength of between 275 and 325 kpsi.

Referring now to FIG. 1, FIG. 2 and FIG. 4, the device 10 further comprises a distracting structure 20 (second component) for distracting the occlusion removing structure 18 to a radially contracted and axially expanded first state from a radially expanded and axially contracted second state. The distracting structure 20 comprises a proximal end 50 and a distal end 52. The proximal end 50 of the distracting structure 20 comprises a second connector 54 configured to mate with the proximal end of the first connector 30. In one embodiment, as shown, the second connector 54 comprises a luer lock-type adaptor as shown, comprising a flared “female” fitting proximally, and a distal “female” fitting with a threaded flange, where the threaded flange of the second connector 54 is configured to mate with the proximal end of the first connector 30, such as the proximal “male” extension of the proximal section 32 of the first connector 30, as shown in the embodiment depicted in the Figures. The second connector 54 can, however, be any suitable connector, as will be understood by those with skill in the art with reference to this disclosure.

The distal end 52 of the distracting structure 20 comprises an elongated tube 56 comprising a proximal end 58, a distal end 60, an axial length defined by the distance between the proximal end 58 and the distal end 60, an outer surface 62 defining an outer circumference, an inner surface 64, and a central lumen defined by the inner surface 64. In a preferred embodiment, the proximal end 58 of the elongated tube 56 is integrally joined to the second connector 54. The outer circumference of the elongated tube is less than the circumference of the inner surface 44 of the tubular mesh 36 when the tubular mesh 36 is in the first state, such as for example between 90% and 99% of the circumference of the inner surface 44 of the tubular mesh 36 when the tubular mesh 36 is in the first state, thereby permitting the elongated tube 56 to fit within the tubular mesh 36 when the device 10 is in the first state.

In a preferred embodiment, the axial length of the elongated tube 56 is at least 90% of the second axial length of the tubular mesh 36 when the device 10 is in the radially contracted and axially expanded first state. In another preferred embodiment, the axial length of the elongated tube 56 is at least 95% of the second axial length of the tubular mesh 36 when the device 10 is in the radially contracted and axially expanded first state. In another preferred embodiment, the axial length of the elongated tube 56 is at least 100% of the second axial length of the tubular mesh 36 when the device 10 is in the radially contracted and axially expanded first state. In another preferred embodiment, the axial length of the elongated tube 56 is at least 105% of the second axial length of the tubular mesh 36.

The elongated tube 56 comprises a material suitable for forcing the tubular mesh 36 into the radially contracted and axially expanded first state by increasing the axial length of the tubular mesh 36 from the first axial length to the second axial length, as will be understood by those with skill in the art with reference to this disclosure. To effect this purpose, in one embodiment, the elongated tube 56 comprises a material having a tensile strength of between 85 and 140 kpsi. In one embodiment, the elongated tube 56 comprises a biocompatible plastic. In a preferred embodiment, the elongated tube 56 comprises a material selected from the group consisting of a braided polyethylene, a braided PVC and a braided nylon derivative to decrease kinking.

Referring now to FIG. 1, FIG. 2, FIG. 3 and FIG. 5, the device 10 further comprises a positioning structure 22 (third component) for positioning the distal end 28 of the occlusion removing structure 18. The positioning structure 22 comprises a proximal end 66 and a distal end 68. The proximal end 66 of the positioning structure 22 comprises a third connector 70 configured to mate with the proximal end of the second connector 54. In one embodiment, as shown, the third connector 70 comprises a “male” extension on the proximal end that functions as an inflation port, and that is connected to a distally tapered “male” distal end, where the tapered “male” distal end is configured to mate with the proximal end of the second connector 54, as shown in the embodiment depicted in the Figures. The third connector 70 can, however, be any suitable connector, as will be understood by those with skill in the art with reference to this disclosure.

In one embodiment, the distal end 68 of the positioning structure 22 comprises a catheter 72 comprising a proximal end 74, a distal end 76, an axial length between the proximal end 74 and the distal end 76, an outer surface 78, an inner surface 80, and an inflation lumen 82 defined by the inner surface 80, and where the catheter further comprises an open proximal end and a cross-sectional area perpendicular to the axial length. The catheter 72 further comprises an inflatable balloon 84 at or near the distal end 76 of the catheter 72. The inflatable balloon 84 has a maximum circumference when inflated. As used in this disclosure, the term “at or near the distal end of the catheter” means that the distal end of the inflatable balloon 84 is positioned within the distal most 10% of the axial length of the catheter 72. For example, if the catheter 72 has an axial length of 10 cm, then the distal end of the inflatable balloon 84 is positioned between 9 cm and 10 cm from the proximal end of the catheter. In another embodiment, the catheter 72 further comprises a blunt-end, flexible, atraumatic tip 86 at the distal end 76 of the catheter 72. In one embodiment, such as when the device 10 is being used in a medical application, the atraumatic tip 86 has an axial length of between 1 cm and 3 cm.

The outer surface 78 of the catheter 72 has a circumference that is smaller than the circumference of the inner surface 64 of the elongated tube 56 of the distracting structure 20, such as for example between 90% and 99%, thereby permitting the catheter 72 to fit within the elongated tube 56 and to slide axially with respect to the elongated tube 56.

In one embodiment, such as when the device 10 is being used in a medical application, the cross-sectional area of the inflation lumen of the catheter 72 is between 1 mm² and 20 mm². In another embodiment, such as when the device 10 is being used in a medical application, the cross-sectional area of the inflation lumen of the catheter 72 is between 2 mm² and 10 mm². In another embodiment, such as when the device 10 is being used in a medical application, the cross-sectional area of the inflation lumen of the catheter 72 is between 3 mm² and 4 mm². The axial length of the catheter 72 is sufficient such that, when the device 10 is assembled, the inflatable balloon 84 extends through the central axial lumen of the cap 48.

In one embodiment, the catheter 72 comprises a material selected from the group consisting of a braided polyethylene, a braided PVC and a braided nylon derivative to decrease kinking. In one embodiment, the inflatable balloon 84 comprises an elastic material, such as for example silicone rubber, latex, a thermoplastic elastomer, such as C-FLEX® (Consolidated Polymer Technologies, Inc.; Clearwater, Fla. US) or a polymer.

Referring now to FIG. 1, FIG. 2 and FIG. 6, in one embodiment, the device further comprises a central stiffening structure 24 (fourth component), as shown particularly in FIG. 6. As can be seen, the central stiffening structure 24 comprises a proximal end 88 and a distal end 90. In one embodiment, the proximal end 88 comprises a sealing member 92 configured to mate with the proximal end of the third connect 70, thereby sealing the proximal end of the central inflation lumen 82. In a preferred embodiment, the sealing member 92 comprises a threaded flange configured to mate with the proximal end of the third connector 70.

The distal end 90 of the central stiffening structure 24 comprises a rod 94 comprising a proximal end 96, a distal end 98, an axial length between the proximal end 96 and the distal end 98, and a surface 100 comprising a circumference. The proximal end 96 of the rod 94 is connected to the sealing member 92. The surface 100 of the rod 94 has a circumference that is smaller than the circumference of the inner surface 80 of the catheter 72, such as for example between 90% and 99%, thereby permitting the rod 94 to fit within the inflation lumen 82 of the catheter 72. In one embodiment, the axial length of the rod 94 is between 10% and 100% of the axial length of the catheter 72. In another embodiment, the axial length of the rod 94 is between 20% and 90% of the axial length of the catheter 72.

In one embodiment, the rod 94 comprises stainless steel, polyethylene or other suitable polymer.

In another embodiment, the present invention is a kit for placing an occlusion removing structure in an open-ended tubular structure comprising a device according to the present invention, and further comprising instructions on how to use the device.

In another embodiment, the present invention comprises a method of placing an occlusion removing structure into a tubular structure with an open distal end, where placing results in the distal end of the occlusion removing structure being positioned at or near the distal end of the tubular structure. In a preferred embodiment, the method comprises providing a device for placing an occlusion removing structure in an open-ended tubular structure according to the present invention.

The method comprises, first, selecting a tubular structure comprising a central lumen and comprising an open distal end with an internal circumference. In one embodiment, the tubular structure comprises an occlusion removing structure within the tubular structure, and the tubular structure is occluded by an occlusion, and the method further comprises removing the occlusion by proximally withdrawing the occlusion removing structure within the tubular structure.

Next, the method comprises providing a device for placing an occlusion removing structure in an open-ended tubular structure according to the present invention having the characteristics as disclosed in this disclosure. The device comprises an occlusion removing structure in a radially contracted and axially expanded first state, a distracting structure and a positioning structure. Then, the distal end of the device is inserted into the proximal end of the tubular structure, and the distal end of the device is advanced through the central lumen of the tubular structure until the inflatable balloon at the distal end of the catheter has advanced completely through the open distal end of the tubular structure. In another embodiment, the distal end of the device is inserted into the tubular structure through a Tuohy Borst adapter.

Then, the method comprises inflating the inflatable balloon until the maximum circumference of the balloon exceeds the internal circumference of the open distal end of the tubular structure. In one embodiment, the device comprises a central stiffening structure according to the present invention, and the method comprises disconnecting the sealing member from the third connector to access the inflation lumen of the catheter to inflate the inflatable balloon.

Next, the method comprises retracting the proximal end of the device proximally until the inflatable balloon contacts the distal end of the tubular structure, thereby limiting any further proximal retraction of the device, and thereby positioning the distal end of the occlusion removing structure at or near the distal end of the tubular structure.

Then, the method comprises deflating the inflatable balloon, and withdrawing the positioning structure from the tubular structure while maintaining the occlusion removing structure in position within the tubular structure. Then, the distracting structure, and the central stiffening structure if present, is withdrawn from the tubular structure while maintaining the occlusion removing structure in position within the tubular structure, thereby causing the occlusion removing structure to change from the radially contracted and axially expanded first state to the radially expanded and axially contracted second state.

In one embodiment, the method further comprises placing the tubular structure within a space or cavity. In a preferred embodiment, the space or cavity is within a human. In one embodiment, the space or cavity is within a non-human animal. In another embodiment, the space or cavity is created by a surgical procedure. In another embodiment, the space or cavity is selected from the group consisting of the abdominal cavity, the bladder, the intestines, the intracranial cavity, the mediastinum, the nasal passages, the stomach, the renal pelvis and the ureter. In another embodiment, the space or cavity is partially or totally created during a surgical procedure on the back, breast, chest, head, hip or vertebral column.

In one embodiment, the proximal end of the tubular mesh extends proximally of the proximal end of the tubular structure, and the method further comprises removing at least some of the proximal end of the tubular mesh that extends proximally of the proximal end of the tubular structure. In a preferred embodiment, the method further comprises attaching the proximal end of the tubular mesh to a cap to secure the proximal end of the tubular mesh.

In another embodiment, the method comprises withdrawing the occlusion removing structure from the tubular structure after the tubular structure becomes occluded with an occluding material, thereby unoccluding the tubular structure.

In one embodiment, the tubular structure is selected from the group consisting of a biliary stent, a biliary tube, a chest tube, a decompression catheter, a feeding tube, a gastrostomy tube, a jejunostomy tube, a mediastinal tube, a nasogastric tube, a nephrostomy catheter, a percutaneous tubular drainage catheter, a peritoneal dialysis catheter, a vascular catheter, and a ventriculostomy tube.

As will be understood by those with skill in the art with reference to this disclosure, this method is particularly useful when the tubular structure has an axial length the dimension of which is unknown by the operator when the device is provided. Further, the method can be used with one device on the tubular structures comprising a variety of axial lengths, where the axial length of the device exceeds the axial length of all of the tubular structures.

Although the present invention has been discussed in considerable detail with reference to certain preferred embodiments, other embodiments are possible. Therefore, the scope of the appended claims should not be limited to the description of preferred embodiments contained in this disclosure.

What is claimed is:

1. A device for placing an occlusion removing structure into a tubular structure with an open distal end, the device comprising:
a) a proximal end, a distal end, and an intermediate portion between the proximal end and the distal end;
b) an occlusion removing structure comprising a proximal end and a distal end, where the proximal end of the occlusion removing structure comprises a first connector, and where the distal end of the occlusion removing structure comprises an elongated tubular mesh comprising a proximal end, a distal end, an axial length defined by the distance between the proximal end and the distal end, an outer surface, an inner surface, and a central lumen defined by the inner surface and further comprising a central lumen cross-sectional area perpendicular to the axial length;
c) a distracting structure for distracting the occlusion removing structure from a radially contracted and axially expanded first state to a radially expanded and axially contracted second state, where the distracting structure comprises a proximal end and a distal end, where the proximal end of the distracting structure comprises a second connector configured to mate with the proximal end of the first connector, and where the distal end of the distracting structure comprises an elongated tube comprising a proximal end, a distal end, an axial length defined by the distance between the proximal end and the distal end, an outer surface defining an outer circumference, an inner surface, and a central lumen defined by the inner surface; and
d) a positioning structure for positioning the distal end of the occlusion removing structure, where the positioning structure comprises a proximal end and a distal end, where the proximal end of the positioning structure comprises a third connector configured to mate with the proximal end of the second connector, where the distal end of the positioning structure comprises a catheter comprising a proximal end, a distal end, an axial length between the proximal end and the distal end, an outer surface, an inner surface, and an inflation lumen defined by the inner surface, and where the catheter further comprises an open proximal end and a cross-sectional area perpendicular to the axial length, and where the catheter further comprises an inflatable balloon at or near the distal end of the catheter, where the inflatable balloon has a maximum circumference when inflated, and where the outer surface of the catheter has a circumference that is smaller than the circumference of the inner surface of the elongated tube of the distracting structure, thereby permitting the catheter to fit within the elongated tube and to slide axially with respect to the elongated tube; and
where the central lumen cross-sectional area in the first state is a first central lumen cross-sectional area, and the axial length in the first state is a first axial length, where the central lumen cross-sectional area in the second state is a second central lumen cross-sectional area, and the axial length in the second state is a second axial length; and
where the first central lumen cross-sectional area is less than the second central lumen cross-sectional area, and the first axial length is greater than the second axial length.

2. The device of claim 1, further comprising a central stiffening structure.

3. The device of claim 1, where the tubular mesh comprises between 5 and 50 strands.

4. The device of claim 1, where the first central lumen cross-sectional area is between 1 mm² and 10 mm².

5. The device of claim 1, where the first axial length is between 10 cm and 100 cm.

6. The device of claim 1, where the second central lumen cross-sectional area is between 10 mm² and 100 mm².

7. The device of claim 1, where the second axial length is between 5 cm and 50 cm.

8. The device of claim 1, where the distal end of the tubular mesh comprises a cap with a central axial lumen with a cross-sectional area sufficient to permit the positioning structure to pass through the central axial lumen of the cap.

9. The device of claim 1, where the proximal end of the elongated tube is integrally joined to the second connector.

10. The device of claim 1, where the outer circumference of the elongated tube is less than the circumference of the inner surface of the tubular mesh when the tubular mesh is in the first state, thereby permitting the elongated tube to fit within the tubular mesh when the device is in the first state.

11. The device of claim 1, where the catheter further comprises a blunt-end, flexible, atraumatic tip at the distal end of the catheter.

12. The device of claim 1, where the distal end of the tubular mesh comprises a cap with a central axial lumen with a cross-sectional area sufficient to permit the positioning structure to pass through the central axial lumen of the cap, and where the axial length of the catheter is sufficient such that, when the device is assembled, the inflatable balloon extends through the central axial lumen of the cap.

13. The device of claim 1, further comprising a central stiffening structure comprising a proximal end and a distal end.

14. The device of claim 1, further comprising a central stiffening structure comprising a proximal end and a distal end, where the proximal end comprises a sealing member configured to mate with the proximal end of the third connector, thereby sealing the proximal end of the central inflation lumen.

15. The device of claim 14, where the distal end of the central stiffening structure comprises a rod comprising a proximal end, a distal end, an axial length between the proximal end and the distal end, and a surface comprising a circumference, where the proximal end of the rod is connected to the sealing member, and where the surface of the rod has a circumference that is smaller than the circumference of the inner surface of the catheter, thereby permitting the rod to fit within the inflation lumen of the catheter.

16. A device for placing an occlusion removing structure into a tubular structure with an open distal end, the device comprising:
a) means for removing an occlusion;
b) means for positioning the distal end of the means for removing an occlusion; and
c) means for distracting the means for removing an occlusion from a radially contracted and axially expanded first state to a radially expanded and axially contracted second state.

17. A kit for placing an occlusion removing structure in an open-ended tubular structure comprising a device of claim 1, and further comprising instructions on how to use the device.

18. A method of placing an occlusion removing structure into a tubular structure with an open distal end, where placing results in the distal end of the occlusion removing structure being positioned at or near the distal end of the tubular structure, the method comprising:
a) selecting a tubular structure comprising a central lumen and comprising an open distal end with an internal circumference;
b) providing a device for placing an occlusion removing structure in an open-ended tubular structure according to claim 1;
c) inserting the distal end of the device into the proximal end of the tubular structure;
d) advancing the distal end of the device through the central lumen of the tubular structure until the inflatable balloon at the distal end of the catheter has advanced completely through the open distal end of the tubular structure;
e) inflating the inflatable balloon until the maximum circumference of the balloon exceeds the internal circumference of the open distal end of the tubular structure;
f) retracting the proximal end of the device proximally until the inflatable balloon contacts the distal end of the tubular structure, thereby limiting any further proximal retraction of the device, and thereby positioning the distal end of the occlusion removing structure at or near the distal end of the tubular structure;
g) deflating the inflatable balloon, and withdrawing the positioning structure from the tubular structure while maintaining the occlusion removing structure in position within the tubular structure; and
h) withdrawing the distracting structure from the tubular structure while maintaining the occlusion removing structure in position within the tubular structure, thereby causing the occlusion removing structure to change from the radially contracted and axially expanded first state to the radially expanded and axially contracted second state.

19. The method of claim 18, where the device further comprises a central stiffening structure comprising a proximal end and a distal end, where the proximal end comprises a sealing member configured to mate with the proximal end of the third connector, thereby sealing the proximal end of the central inflation lumen, and where the method further comprises disconnecting the sealing member from the third connector to access the inflation lumen of the catheter to inflate the inflatable balloon.

20. The method of claim 19, further comprising withdrawing the central stiffening structure from the tubular structure.

21. The method of claim 18, where the tubular structure is selected from the group consisting of a biliary stent, a biliary tube, a chest tube, a decompression catheter, a feeding tube, a gastrostomy tube, a jejunostomy tube, a mediastinal tube, a nasogastric tube, a nephrostomy catheter, a percutaneous tubular drainage catheter, a peritoneal dialysis catheter, a vascular catheter, and a ventriculostomy tube.

22. The method of claim 18, further comprising placing the tubular structure within a space or cavity.

23. The method of claim 22, where the space or cavity is selected from the group consisting of the abdominal cavity, the bladder, the intestines, the intracranial cavity, the mediastinum, the nasal passages, the stomach, the renal pelvis and the ureter.

24. The method of claim 22, where the space or cavity is partially or totally created during a surgical procedure on the back, breast, chest, head, hip or vertebral column.

25. The method of claim 18, where the proximal end of the tubular mesh extends proximally of the proximal end of the tubular structure, and where the method further comprises removing at least some of the proximal end of the tubular mesh that extends proximally of the proximal end of the tubular structure.

26. The method of claim 18, further comprising withdrawing the occlusion removing structure from the tubular structure after the tubular structure becomes occluded with an occluding material, thereby unoccluding the tubular structure.
